(19) United States
(12) Patent Application Publication
(10) Pub. No.: US 2025/0069193 A1
(43) Pub. Date: Feb. 27, 2025

(54) A METHOD FOR GENERATING QUANTITATIVE MR RELAXATION MAPS AND SUBSEQUENT MR IMAGE SYNTHESIS

(71) Applicant: ARAMIS IMAGING LLP, TRIVANDRUM, KERALA (IN)

(72) Inventor: Ganesh S, KOLLAM, KERALA (IN)

(73) Assignee: ARAMIS IMAGING LLP, TRIVANDRUM, KERALA (IN)

(21) Appl. No.: 18/728,642

(22) PCT Filed: Jan. 18, 2023
(86) PCT No.: PCT/IN2023/050055

§ 371 Date: Jul. 12, 2024

(30) Foreign Application Priority Data
Jan. 19, 2022 (IN) 202241003130

(51) Int. Cl. G06T 5/50; G01R 33/56; G06T 7/00; G06T 7/62

(52) U.S. Cl. CPC G06T 5/50; G01R 33/5608; G06T 7/0012; G06T 7/62; G06T 2207/10088; G06T 2207/20212; G06T 2207/30004

(57) ABSTRACT

The present invention relates to a method for producing MRI images with the following features: Magnetic resonance images are acquired using an MRI system. These images can be used to determine the relaxation times of protons and can be displayed as parametric maps (110, 111, 112). Various other contrasts can be synthesized by combining T1, T2 and PD values and varying the scan parameters using Bloch equations or modifications (104). The images thus synthesized will be displayed in the appropriate display. The entire process can be implemented even as a client-side software programme or even as a web based application.

### FIELD OF INVENTION

This invention comes under the field of medical radiology more specifically, to the field of Magnetic Resonance Imaging or MRI technology and data processing and visualization specially adapted for MR. This invention relates to a method for enhancing and improving the visualization and information regarding tissues obtained from a magnetic resonance scan by using a computer software.

### BACKGROUND OF THE INVENTION

MRI (Magnetic Resonance Imaging) is a non-invasive radiological technology that uses strong magnetic fields and radio waves to produce anatomical and physiological images of the body that cannot be seen on studies using X-Rays/CT or Ultra-Sound scans. It enables therapists and doctors to have a view of the internal regions of bones/cartilages/joints/organs and enable them to study, detect, diagnose and treat patients affected with health issues like strokes, neurological disorders, tumours, orthopaedic problems and much more. MRI is a part of Nuclear Magnetic Resonance (NMR) which can be used for imaging in other applications like NMR spectroscopy.

When placed in an external powerful oscillating magnetic field, certain atomic nuclei are able to absorb radio frequency energies and produce a spin polarization which can induce a Radio Frequency (RF) signal in a RF coil which can be used to study structures. MRI technique utilizes the hydrogen atoms present abundantly in living organisms for MRI diagnostic purposes. By varying the pulsing sequence of the RF energies, images with different contrasts of the tissues under study can be obtained.

Depending on the other atoms bonded to the hydrogen atoms, it is possible to differentiate the different compounds. The RF coils are switched on and off repeatedly with varying time and pulse impulses to obtain a continuous image reading. A gradient system is used to localize the scan region. MRI sequencing refers to a set of RF pulses with specific timed pulses and gradients to result in a certain image appearance.

MRI visualizes cross-sectional images of any body part or object that contains hydrogen nuclei, which chemically represents a proton, in any desired plane. The electron orbiting around the proton of the hydrogen atom generates a magnetic field, that can be considered analogous to a bar magnet. When subjected to a strong external magnetic field (termed BO), these align in either a parallel or anti-parallel direction to it and undergo angular rotation (spin), similar to a spinning top (termed precession), forming a net magnetic vector. The frequency of precession (angular momentum) is called the Larmor frequency and is given by the equation, ω=yB

### Where

ω is the Larmor frequency in MHz
y is the gyromagnetic ratio in MHz/tesla and
B is the strength of the magnetic field in Tesla

Subsequent application of a radiofrequency pulse of specified frequency and amplitude, influences the spin of the protons and causes it to change direction because of acquiring energy. On switching off the pulse, the protons return to their original state and give off the excess energy, which can be obtained as a signal (echo). The entire process can be repeated several times to obtain as much information as possible from the object being imaged. The time between application of the radio frequency pulse and the received signal is called time-to-echo (TE) and the time interval between two radiofrequency pulses is called time-to-repetition (TR). The angle by which the protons change direction (flip) is determined by the duration and amplitude of the RF pulse with usual angles being in multiples of 90 degrees. However, smaller angles can also be used. These parameters of TE, TR and flip angles are parameters that are entered into the MR scanner at the time of image acquisition.

The excitation-deexcitation cycle during the application and removal of the RF pulse causes the protons to change directions, while at the same time continuing to precess. The change in direction causes a concomitant shift in the net magnetization vector followed by reversion to its original state. This represents a collective shift in the direction of the spinning protons and gives rise to two numerical entities called “relaxation time(s)”. T1 or longitudinal relaxation is the time taken for the flipped magnetic vector to return to 63% of its original magnitude in its original direction. This is accompanied by a simultaneous reduction of the magnitude in the flipped plane, which, when it reaches 37% of its initially acquired valued, gives us T2. T1 is dependent on the interaction between the proton spins and the surrounding tissue and thus is called spin-lattice relaxation. T2 is dependent on the rapid dephasing of protons due interaction between adjacent proton spins and is called spin-spin relaxation. It is denoted as T2*.

After excitation by the independent relaxation processes of T1 (magnetization in the same direction as the static magnetic field) and T2 (spin-spin; transverse to the static magnetic field) every tissue returns to its relaxed state. A T1-weighted image is created by changing the repetition time and allowing the magnetization to recover. This is useful for obtaining morphological data and post-contrast imaging. A T2-weighted image is formed by varying the echo time, thereby allowing the decay of the magnetization. This is useful for detecting swelling, inflammation, lesions, and the reproductive organs.

T1 and T2 are tissue specific, meaning that they are an inherent property of the tissue being imaged. For e.g., T1 and T2 relaxation times are different for fat, muscle, and water. By leveraging this property, the input parameters can be adjusted in the MR scanner (i.e., TE, TR, and flip angle) to bring out tissue differences on a gray scale image as differences in signal intensity. Thus, by applying “weighting” an image can be made to show more T1 signal or T2 signal, thus indirectly brightening or darkening a particular tissue on a gray scale display. It is this weighting that provides image contrast between different tissues. So, input parameters influence or determine, how well the contrast of a particular tissue appears on the gray scale image, or in other words the signal intensity.

There are many combinations of scan parameters, that can be used to produce MR images from a scanner. A fast method among them is fast spin-echo (FSE)

The signal intensity of a pixel, corresponding to a voxel of tissue, of a fast spin-echo image is given by the equation:

$$S = PD . exp ( - TE / T 2 ) . ( 1 - exp ( - ( TR - ETL . ESP ) / T 1 ) )$$

Where ETL is the echo train length and ESP denotes the spacing of the echoes.
The equation for an inversion recovery sequence is

$$S = PD . exp ( - TE / T 2 ) . ( 1 - 2 exp ( - TI / T 1 ) + exp [ ( - ( TR - ETL . ESP ) / T 1 ) ) ]$$

And for a double inversion recovery sequence is

$$S = PD . exp ( - TE / T 2 ) . ( 1 - 2 exp ( - TI 1 / T 1 ) +$$

$$2 exp ( - TI 2 / T 1 ) - exp ( - ( TR - ETL . ESP ) / T 1 ) )$$

Magnetic resonance images are acquired using a magnetic resonance scanner. These images are then used to determine relaxation times T1, T2 and scaled Proton Density (PD) of each pixel. These properties can be displayed as parametric maps. Various other contrasts can be synthesized combining T1, T2 and PD values and varying scan parameters using Bloch equations, such as time-to-echo (TE), time-to-repetition (TR), inversion time (Tl) and flip angle (a). The scanner parameters can be precisely adjusted by numerical input to specific controls displayed on a computer screen or using mouse clicks on controls which represent combinations of these parameters. The images thus synthesized will be displayed in a window on the screen.

Study scan times are dependent on protocols that list out the sequences to be done with regards to a particular body part or pathological process. In several situations, multiple additional sequences need to be acquired, that will help in better delineation of tissues that are specific to the body part being imaged. Although, this contributes to better diagnosis and translates into better patient care, it increases the time required for the scan and indirectly contributes to the healthcare costs, by decreasing utilizable time of the machine.

An MR study must be tailored to its clinical indication and requires considerable technical skill. A typical MR study consists of sequences, which are a combination of scanner parameters, that brings out the best soft tissue resolution for the anatomic part and/or pathology being images. The acquisition of multiple sequences requires more time and machine utilization, which can reduce patient throughput and potentially increase indirect healthcare costs.

A few patents related to this field are discussed below:

The patent U.S. Pat. No. 10,345,414B2 (Rapid quantitative abdominal imaging with magnetic resonance fingerprinting (MRF)) relates to a method of acquiring fingerprints of the abdominal region by using fast imaging pulse sequences and creating a field map and producing corrected quantitative values using which a quantitative image can be created.

The patent US20190265322A1 (Diffusion-Weighted Double-Echo Magnetic Resonance Fingerprinting (MRF)) relates to a method of obtaining different MRI signals by variably sampling the time and echo pulse sequence points related to a particular tissue so that different points of study can be obtained.

The patent CN107072595B (Adaptive re-planning based on multi-modality imaging) relates to a system or method of producing a composite image from MRI data by applying corrections and generating a relaxation map for 4D MR image data using an inverse conversion process for use in radiation therapy.

Our present system offers a low-cost solution to obtain fast quantitative MRI images by synthesizing MR images corresponding to sequences acquired conventionally using different scan parameters from acquiring 3 standard artefact free MR sequences. Historical or early study data can also be used in the production of the MRI images.

### OBJECTIVE OF THE INVENTION

The main objective of our system is to provide a fast solution to obtain quantitative MR relaxation maps from routine MR sequences.

Another objective of our system is to provide a method of obtaining MRI images using any type of datasets including historical datasets to quantify the presence of a particular type of tissue within suspected pathology.

Another objective of our system is to synthesize MR images of different contrast using the quantitative data.

### SUMMARY OF THE INVENTION

The following summary is provided to facilitate a clear understanding of the new features in the disclosed embodiment, and it is not intended to be a full, detailed description. A detailed description of all the aspects of the disclosed invention can be understood by reviewing the full specification, the drawing and the claims and the abstract, as a whole.

The major task addressed by the present invention is time efficient production of quantitative MR images from acquiring 3 standard artefact free MR sequences having different scan parameters. This can be done on historical or previous study data as well.

The present invention uses a numerical input method for varying scan parameters while synthesising MR images of varying contrast, which is more accurate. This method can be used with any suitable dataset, irrespective of when it was acquired, meaning that the method can be applied on historic data as well. Using the present invention, the presence of a particular type of tissue can be theoretically quantified and characterised.

### BRIEF DESCRIPTION OF THE DRAWINGS

The manner in which the proposed system works, briefly summarized above and described in detail below, may be had by reference to the components, some of which is illustrated in the appended drawing It is to be noted; however, that the appended drawing illustrates only typical embodiments of this system and are therefore should not be considered limiting of its scope, for the system may admit to other equally effective embodiments.

Throughout the drawings, the same drawing reference numerals will be understood to refer to the same elements and features.

The features and advantages of the present proposed system will become more apparent from the following detailed description along with the accompanying figures, which forms a part of this application and in which:

FIG. 1: Block Diagram describing the components and the workflow of our system in accordance with our present invention.

FIG. 2: Block Diagram describing the filtering of tissues and measurement of volume processes of our method, in accordance with our present invention.

### REFERENCE NUMERALS

100 Start process of parametric maps calculation with synthesis of images of different contrast
101 Set of images with specific TE and TR (T1W)
102 Set of images with specific TE and TR (T2W)
103 Set of images with specific TE and TR (PD or other weighting)
104 Obtaining the value of each pixel at same position on all three sets and using it for partial reduction of Bloch equations
105 Finding any variable T1, T2 or scaled PD using fast iterative approximation or substitution in partially solved equations
106 Finding other two variables by substitution
107 T2
108 Tl
109 sPD
110 Tl
111 TE
112 TR
113 Pre-set with common TE, TR and Tl values selected by user
114 G-Substitution of Bloch value equations to synthesize images of various signal entities
115 H—Render three variables with colour scaling
116 I—Display synthesized images in grey scale
117 J—Display as colour image
201 Obtaining T1, T2 range of specific tissue from parametric maps or previous known values. 11 and h1 and 12 and h2 are the lower and upper values for T1 and T2
202 Obtaining pixels only in the range of T1 and T2
203 Display of information
204 Obtain number of pixels and calculating the volume using information from DICOM metadata

### DETAILED DESCRIPTION OF THE INVENTION

The principles of operation, design configurations and evaluation values in these nonlimiting examples can be varied and are merely cited to illustrate at least one embodiment of the invention, without limiting the scope thereof.

The embodiments disclosed herein can be expressed in different forms and should not be considered as limited to the listed embodiments in the disclosed invention. The various embodiments outlined in the subsequent sections are construed such that it provides a complete and a thorough understanding of the disclosed invention, by clearly describing the scope of the invention, for those skilled in the art.

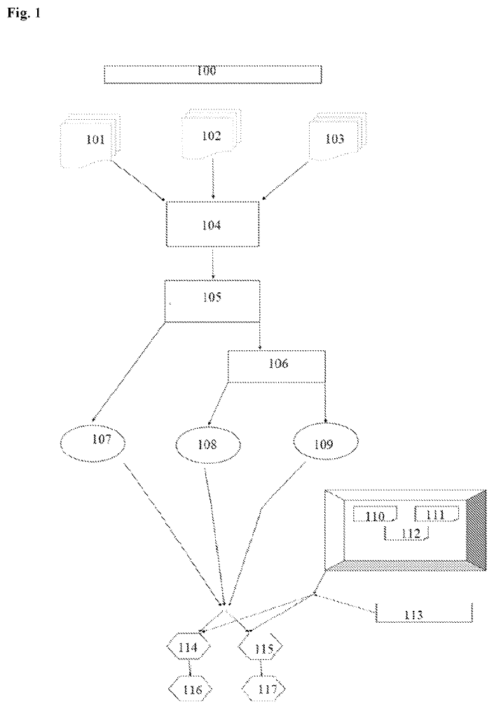

This image FIG. 1 relates to a method of calculating with parametric maps with synthesis of images from different contrast. Sets of images with specific TE and TR (T2W) (T1W, T2W PDW or other similar sequences). The value of each pixel at the same position on all three sets are obtained and used for partial reduction of Bloch equations (104). Finding any variable T1, T2 or scaled PD using fast iterative approximation or substitution in partially solved equations (105). T2, Tl and sPD (107, 108, 109) are used to solve the equations. Numerical input from the user is displayed on a computer screen (110, 111, 112). G-Substitution of Bloch value equations to synthesize images of various signal intensities (114). H-Render three variables with colour scaling (115), I-Display synthesized images in grey scale (116), J-Display as colour image (117).

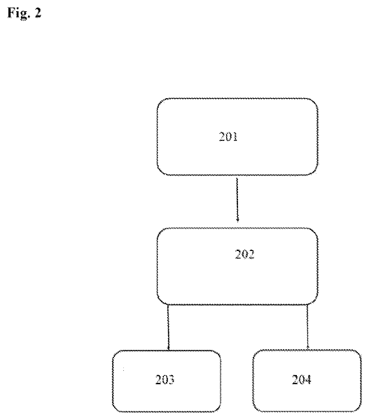

The FIG. 2 describing the filtering of tissues and measurement of volume processes of our method, in accordance with our present invention is shown. Obtaining T1, T2 range of specific tissue from parametric maps or previous known values (200 and 201). 11 and hl and 12 and h2 are the lower and upper values for T1 and T2, obtaining pixels only in the range of T1 and T2, display of information (2,3) (203), obtain number of pixels and calculating the volume using information from DICOM metadata (204)

In one embodiment of our invention, Magnetic resonance images are acquired using a magnetic resonance scanner. These images are then used to determine relaxation times T1, T2 and scaled proton density of each pixel. These properties can be displayed as parametric maps. Various other contrasts can be synthesized combining T1, T2 and PD values and varying scan parameters using Bloch equations, such as time-to-echo (TE), time-to-repetition (TR), inversion time (Tl) and flip angle (a). The scanner parameters can be precisely adjusted by numerical input to specific controls displayed on a computer screen or using mouse clicks on controls which represent combinations of these parameters. The images thus synthesized will be displayed in a window on the screen.

In one embodiment of our invention, a computer program, which in this case serves as the apparatus for viewing medical images. The invention uses pre acquired MR images with no movement artefact to rapidly generate quantitative MR parametric maps with subsequent synthesis and display of MR images with filtering, enhancement, qualification and quantification of tissues.

In one embodiment of our invention, Pre-acquired MR images with no or minimal inter or intra sequence movement with three varying scanner parameters are processed using a fast algorithm that will calculate the T1 and T2 relaxation times and scaled Proton Density of each pixel in the image(s). The current algorithm employs rapidly converging iterative methods for a precise approximation, similar to binary search. A direct arithmetic operation can also be used for this. As the algorithm is fast, the process can be performed even in a universally available web browser without use of other specialized software or hardware. The method is mostly independent of the vendor of the scanner and the sequences include, routinely employed ones. It does not require acquisition of special or tailored sequences. The method can also be applied to images acquired in the past or in previous studies.

In another embodiment of our invention, the calculated relaxation times can be inserted into the standard equations for SI and the parameters TE, TR, TI and a can be numerically entered using either type keys or mouse clicks on a control which represents a combination of scanner parameters, displayed on the computer screen to generate images of varying contrast. Entering numerical values are advantageous as the user has more precise and rapid control over the setting of scan parameters.

In one embodiment of our invention, the parametric maps can be displayed based on one or more of relaxation times. In addition, a colour map image can be rendered using T1, T2 and PD in place of R, G and B colour components.

In one embodiment of our invention, by using techniques to synthesize MR images of varying contrasts, scan times can be reduced for patients and facilitate faster patient throughput. This will indirectly reduce health care costs.

In another embodiment of our invention, it can facilitate rapid viewing of images to further decide on tailoring of protocols in an emergency setting.

According to one embodiment of our invention, as specific tissues can have specific Tl, T2 and PD values, these tissues can be potentially identified, isolated, enhanced or suppressed. This may help in accurately identifying tissues or pathological processes.

According to one embodiment of our invention, as we can alter the scan parameters virtually this method might be useful in MR sequence design.

The advantages of the present invention are:
The entire process can be implemented even as a client-side software programme on the internet browser (web-based application) for rapid deployment and easy scaling up. Hence it can be deployed as a web-based application.
Numerical input possible making it extremely accurate
Can work on historical data
Characterization and quantification of tissue theorectically possible

While the foregoing written description of the invention enables one of ordinary skill to make and use what is considered presently to be the best mode thereof those of ordinary skill will understand and appreciate the existence of variations, combinations, and equivalents of the specific embodiment, method, and examples herein. The invention should therefore not be limited by the above described embodiment, method, and examples, but by all embodiments and methods within the scope of the invention as claimed.

We claim:

1. A method for processing MRI images comprising of:
magnetic resonance images acquired using an MRI system, the said images are used to determine relaxation times T1, T2 and scaled proton density of each pixel and displayed as parametric maps (200 and 201).
contrasts synthesized by combining T1, T2 and PD values and varying the scan parameters using Bloch equations (104).
the said images thus synthesized is displayed in the display (110, 111, 112).

2. The method for producing MRI images, as claimed in claim 1, wherein, the said scanner parameters is precisely adjusted by numerical input to specific controls displayed on a display or using mouse clicks on controls which represent combinations of these parameters.

3. The method for producing MRI images, as claimed in claim 1, wherein, the said scanner parameters is numerically entered using the appropriate input device, to provide integration with historical MRI data and other MRI data.

4. The method for producing MRI images, as claimed in claim 1, wherein, the said method is implemented even as a client-side software programme on the internet browser as a web-based application.

5. The method for producing MRI images, as claimed in claim 1, wherein, the said method is implemented as a potential tool for characterization of the tissue being imaged on a pixel-by-pixel basis, by varying image contrast and bringing out the difference between native and acquired MRI parameters.

6. The method for producing MRI images, as claimed in claim 1, wherein, the said method obtains the number of pixels and calculates the volume using information from DICOM metadata (204).

7. The method for producing MRI images, as claimed in claim 1, wherein, the said method provides quick synthesis of MR images of varying contrasts by employing rapidly converging iterative algorithms.

8. The method for producing MRI images, as claimed in claim 1, wherein, the said method on pre acquired MR images with no movement artefact to rapidly generate quantitative MR parametric maps with subsequent synthesis and display of MR images with filtering, enhancement, qualification and quantification of tissues.
